(19) United States
(12) Patent Application Publication
(10) Pub. No.: US 2022/0233801 A1
(43) Pub. Date: Jul. 28, 2022

(54) NASAL CANNULA WITH A BETTER-SECURED CONNECTION TO A SUPPLY LINE

(71) Applicant: HAMILTON MEDICAL, AG, Bonaduz (CH)

(72) Inventors: Martin SUCCETTI, Scharans (CH); Sandro WALKER, Chur (CH)

(21) Appl. No.: 17/615,708

(22) PCT Filed: Jun. 4, 2020
(86) PCT No.: PCT/EP2020/065563

§ 371 Date: Dec. 1, 2021

(30) Foreign Application Priority Data
Jun. 7, 2019 (DE) 10 2019 115 639.4

(51) Int. Cl. A61M 16/06; A61M 16/08

(52) U.S. Cl. CPC A61M 16/0672; A61M 16/0816; A61M 2202/0208; A61M 16/0875; A61M 16/0683

(57) ABSTRACT

The invention relates to a patient treatment gas supply system having a nasal cannula having a cannula connection and a treatment gas outlet opening; a treatment gas supply line having a line connection which is designed to form a detachable treatment-gas-carrying connection in a connection region with the cannula connection, wherein the cannula connection and the line connection overlap along a flow path formed via the connection region; and a blocking assembly which can be moved between a blocking position and a release position, wherein a force necessary for releasing the connection between the cannula connection and the line connection is greater when the blocking assembly is in the blocking position than when the blocking assembly is in the release position.

The present invention concerns a patient therapeutic gas supply system, comprising a nasal cannula for the nasal supply of a patient with therapeutic gas, wherein the nasal cannula comprises: A cannula connector, and a therapeutic gas dispensing aperture for dispensing the therapeutic gas to the patient, wherein the nasal cannula defines a fluid conduit between the therapeutic gas dispensing aperture and the cannula connector; a therapeutic gas supply line for feeding therapeutic gas to the nasal cannula, wherein the therapeutic gas supply line exhibits a line connector which is set up so as to form a separable therapeutic gas-carrying connection in a connecting region with the cannula connector, wherein once the therapeutic gas-carrying connection has been established, the cannula connector and the line connector overlap along a section of a flow path proceeding through the connecting region; and a locking module, wherein the locking module is movable between a locked position and a released position, wherein when the locking module is in the locked position, a force needed for separating the connection between the cannula connector and the line connector is quantitatively larger than when the locking module is in the released position.

A flow path which is always a virtual flow path can be defined in this application through the path which connects the geometric centers of gravity of the cross-sections of the therapeutic gas-carrying regions, e.g. of the inner volume of a hose section and/or of the nasal cannula, of the patient therapeutic gas supply system along the flow movement. In particular, in case of doubt the flow path can be conceived as penetrating centrally through the therapeutic gas-carrying fluid conduits of the patient therapeutic gas supply system.

Patient therapeutic gas supply systems are described in this application in a state in which the therapeutic gas-carrying connection is formed, unless expressly described otherwise.

In addition, describing the directions relies on using the flow path for a flow path direction as a reference wherein a flow path direction proceeds in parallel or antiparallel to a direction vector describing a tangent of the flow path. A radial direction relative to the flow path should be understood as a direction which proceeds perpendicularly to a tangent of the flow path at a point of the flow path and points away from this point. A circumferential direction denotes a direction encircling the flow path or an axis. In the case of a curved flow path, this leads to a change in the flow path's direction, the radial direction, and the circumferential direction in a Cartesian coordinate system fixed relative to patient therapeutic gas supply system.

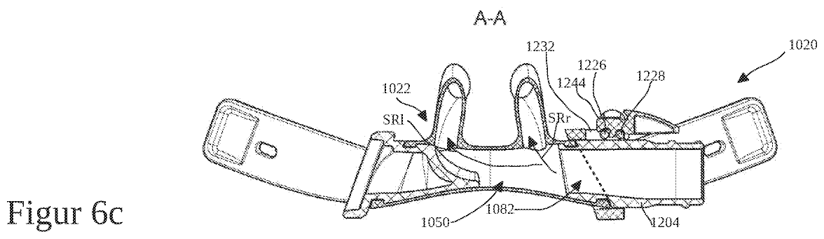

A patient therapeutic gas supply system as described above is known for example from the publication WO 2014/142681, there an embodiment of FIGS. 15E to 15G. In this state of the art, the locking module is configured in the form of two tongues, each provided with a projection, which are configured at the line connector. If the line connector is plugged into the cannula connector, the projections engage behind an annular shoulder and impede the separating of the therapeutic gas-carrying connection. Due to the proximity of the annular shoulder to the projections carrying the therapeutic gas-dispensing aperture, however, it is cumbersome to separate the connection between the cannula connector and the line connector, since the user, e.g. the patient or the patient's caregiver, has to position his or her finger accurately on the nasal cannula in order to press the projections of both tongues inward while at the same time pulling the line connector away from the nasal cannula. This can, moreover, result in the tongues not being pressed inward deeply enough and the released position thereby inadvertently not reached. If an attempt is then made to separate the therapeutic gas-carrying connection, the nasal cannula can be unintentionally removed which can be very uncomfortable for the patient.

It is, therefore, the task of the present invention to provide a patient therapeutic gas supply system in which a separable and secure therapeutic gas-carrying connection can be configured.

This task is solved according to the invention by means of a patient therapeutic gas supply system of the type mentioned at the beginning, in which once the therapeutic gas-carrying connection has been made, the locking module is displaceable between the locked position and the released position relative to the cannula connector and to the line connector in such a way that it exhibits in the locked position a greater overlap with the connecting region than in the released position. Due to the greater overlap of the locking module with the connecting region, freedom of movement of the cannula connector and/or of the line connector is more strongly restricted in the connecting region in the locked position than in the released position, such that deformation of the cannula connector and/or of the line connector, which can lead to separating of the therapeutic gas-carrying connection, is counteracted such that the security of the therapeutic gas-carrying connection is enhanced.

Preferably, the locking module is located further out in the radial direction relative to the flow path than the cannula connector and the line connector. The overlap of the locking module with the connecting region then serves for the user as an indicator for the presence or absence of the locked position, whereby the frequency of misjudgments as to whether the locking module is in the locked position is decreased, which in turn simplifies separating the therapeutic gas-carrying connection. Furthermore, changing between the locked position and the released position by displacing the locking module allows the user to change over easily between the two positions. If the locking module is in the locked position, then due to the described forces needed for separating the therapeutic gas-carrying connection, an especially secure therapeutic gas-carrying connection is formed.

The connecting region can extend in the flow path direction beyond the region defined by the overlapping of the cannula connector and line connector. Preferably, the connecting region is only that region in which the cannula connector and line connector extend over a common flow path section, i.e. overlap.

In order to form the tightest possible therapeutic gas-carrying connection, both the line connector and the cannula connector each exhibit coupling sections completely encircling the flow path. This applies in particular to those connection sections of the line connector and cannula connector which overlap when forming the therapeutic gas-carrying connection.

In order to facilitate the making of a permanently tight therapeutic gas-carrying connection, preferably one connector out of the line connector and cannula connector is configured as a deformation connector with at least in the connection section lower stiffness against expansion in the radial direction than the respective other connector. The respective other connector consequently exhibits as a supporting connector, at least in its connection section, higher stiffness against deformation orthogonally to the flow path.

Preferably, the different stiffnesses are achieved by using materials with different modules of elasticity for the cannula connector and the line connector. In order to make the overlapping connection, the deformation connector can under radial expansion be pushed or pulled against its material elasticity over the supporting connector. When back in its relaxed shape under the pre-tensioning effected by its expansion deformation, the deformation connector then abuts on the supporting connector with a sealing effect.

Since the locking module is preferably arranged radially outside the line connector and cannula connector to facilitate actuation, the locking module arranged radially outside in this way can in addition act on the deformation connector located nearer to it radially so as to advantageously enhance the sealing effect and/or the connection strength between the line connector and cannula connector in the connecting region, for instance by exerting a mechanical force on the deformation connector that is thus directly accessible to the locking module.

Since the nasal cannula is or can come almost constantly in contact with the skin of the patient during therapeutic gas supply, preferably the cannula connector is the aforementioned deformation connector.

At this point let it be made clear that for configuring the separable therapeutic gas-carrying connection, a friction coupling and/or a positive-locking coupling and/or a firmly bonded coupling contributes to and/or is the connection.

In order to transmit forces across as large an area as possible to the connecting region, thus contributing to secure configuration of the therapeutic gas-carrying connection, the locking module surrounds the flow path at least section-wise in a circumferential direction, preferably completely in the circumferential direction.

In a preferred embodiment, the locking module is configured as separate from (i.e. not integral with) the cannula connector and the line connector. As a result, the material or materials of the locking module can be chosen independently from the materials of the cannula connector and of the line connector. In order to ensure proper operation of the patient therapeutic gas supply system, the locking module is installed misplacement-proof on the cannula connector or the line connector in at least one, preferably in both positions out of the locked position and released position.

In an especially preferred embodiment, when the locking module is in the locked position the locking module pre-tensions at least section-wise a connector out of the cannula connector and the line connector in the direction of the other connector out of the cannula connector and the line connector more strongly than when the locking module is in the released position. As a result, in the locked position there can act in the overlap region, in particular also due to the greater overlap of the locking module with the connecting region in the locked position of the locking module compared with the state of the locking module in the released position, a quantitatively greater force between two mutually abutting surfaces of the cannula connector and of the line connector than when the locking module is in the released position. A frictional force resulting from same between these two surfaces or a force resulting from same and counteracting a deformation of a connector out of the cannula connector or line connector has to be overcome when separating the therapeutic gas-carrying connection, which in a simple manner increases the force needed for separating the connection between the cannula connector and the line connector compared with the state in which the locking module is in the released position.

The locking module comprises a locking section, which when the locking module is in the locked position overlaps with the connecting region. The locking section can be the locking module. It is, however, preferable for the locking module in addition to the locking section to comprise a carrier section, which when the locking module is in the locked position does not overlap with the connecting region. Preferably the locking section and the carrier section are configured integrally.

The locking section can comprise a plurality of part-sections arranged around an axis penetrating through the connecting region, which are arranged in the circumferential direction around the axis spaced from one another and with preferably equal angles between neighboring part-sections around the axis. The axis preferably coincides with the flow path or one of its tangents or is at least configured parallel to one of these tangents. As a result, the weight of the locking module can be reduced. It is, however, preferable for the locking section to be configured as continuous in the circumferential direction.

The locking section can comprise a component U-shaped at least in one aspect, and/or a partly or completely closed reversibly deformable band and/or an essentially rigid annular element, such that a locking section matched to the constraints such as flexibility of the cannula section and/or of the duct section, spatial restrictions for the movement of the locking section etc. can be provided.

In order to contribute to the forming of a securely and safely manageable therapeutic gas-carrying connection, the patient therapeutic gas supply system can further comprise at least one positive-locking arrangement which exhibits a positive-locking formation configured at the line connector and a positive-locking counter-formation configured at the cannula connector, wherein the positive-locking formation at least contributes to the configuration of the separable therapeutic gas-carrying connection by forming positive locking between the positive-locking formation and the positive-locking counter-formation. As a result, in contrast to the presence of a therapeutic gas-carrying connection relying solely on friction coupling, a connection type can be provided which provides the user with tactile and/or acoustic feedback when forming or separating the positive locking, when for example the positive-locking formation interacts with the positive-locking counter-formation or is separated from the latter, such that the user can determine with enhanced certainty whether a therapeutic gas-carrying connection is made or separated. The positive-locking arrangement is preferably configured in the connecting region.

In the released position of the locking module there is preferably no overlap of the locking module and/or of the locking section with the connecting region and/or the positive-locking arrangement.

In order to secure the therapeutic gas-carrying connection more strongly against unintentional separating, the positive-locking arrangement can surround the flow path at least in part, preferably completely.

A pair of a positive-locking formation and a positive-locking counter-formation, which are at least part of the positive-locking arrangement or form the latter, can be a pair consisting of a groove and a spring, and/or a pair consisting of a projection and a recess, and/or a pair consisting of a claw and an edge which is engaged from behind by the claw, and/or a pair consisting of tooth systems engaging with one another, and/or a pair consisting of a pawl and a surface provided with retaining teeth, or a similar pair.

Preferably the positive-locking arrangement is so configured that when the locking module is in the released position, a displacement movement of a displacement section of the positive-locking formation or of the positive-locking counter-formation along a displacement path contributes to the separating of the positive locking. It is possible for the displacement movement of the displacement section to be accompanied by a deformation of the displacement section or its surroundings, which defines a displacement deformation. Impeding the displacement movement is understood to be any quantitative increase in a force needed for performing the displacement movement compared with performing the displacement movement without the conditions or features constituting the impeding. An at least partial prevention of the displacement movement is a special case of a greatest possible impeding of the displacement movement. Because of the facilitated deformability, the displacement section is preferably configured at the deformation connector.

In a preferred embodiment, at least one formation out of the positive-locking formation and the positive-locking counter-formation proceeds at least section-wise non-parallel to a normal plane of the flow path which normal plane intersects the one formation out of the positive-locking formation and the positive-locking counter-formation. This makes it possible for only one or a predetermined plurality of predetermined relative positions to be taken up between the cannula connector and the line connector when the therapeutic gas-carrying connection is made, and consequently the patient therapeutic gas supply system can in a straightforward manner by means of a thus configured indexing mechanism take up one or a plurality of predetermined arrangements comfortable for the patient.

In an especially preferred embodiment, the patient therapeutic gas supply system for securing the therapeutic gas-carrying connection further comprises a position-securing device, wherein in the locked position the locking module can be secured by means of a position-securing device against movement in the direction of the released position. Any impeding of a movement of the locking module from the locked position in the direction of the released position counts as securing. What has been said above about impeding the displacement movement, applies correspondingly to impeding the aforementioned movement of the locking module. Here it should be allowed for the locking module to exhibit backlash in the locked position, although backlash-free securing is preferred.

According to an advantageous further development, the position-securing device can be moveable between a secured position and a released position. Preferably when the locking module is in the locked position and the position-securing device is in the secured position, with the assistance of the position-securing device a physical barrier is formed which prevents movement of the locking module from the locked position to the released position. Moreover when the position-securing device is in the released position, the physical barrier formed with the assistance of the position-securing device impedes the movement of the locking module from the locked position to the released position less strongly than when the locking module is in the locked position and the position-securing device is in the secured position. Such a position-securing device in combination with the locking module reduces the likelihood of unintentional separating of the therapeutic gas-carrying connection more strongly than a patient therapeutic gas supply system with only a locking module since due to the impeding described above, in the secured position the position-securing device reduces the likelihood that the locking module is unintentionally moved from the locked position to the released position.

For straightforward, preferably self-activating securing of the locking module in the locked position, the position-securing device can comprise a snap-in arrangement with a snap-in projection which is pre-tensioned in the direction of a snap-in seat of the snap-in arrangement.

An extreme case of strong impeding when the locking module is in the locked position and the position-securing device is in the secured position, is the forming by means of the physical barrier of a barrier that cannot be overcome non-destructively.

A snap-in seat can be configured as a concave section of a surface, in particular as a material recess, an indentation, or a concave spatial section bounded by projections.

The physical barrier is preferably a barrier that can be overcome non-destructively.

In an especially simple configuration, the position-securing device comprises a snap-in arrangement with a snap-in element and a mating snap-in element. Again it is preferable that the snap-in arrangement forms a latching mechanism that can be overcome non-destructively. The snap-in arrangement can contribute to the forming of the physical barrier or form it.

In order to reduce the error-proneness of the effect of the snap-in arrangement, the snap-in element is arranged at a component out of the locking module and either the line connector or the cannula connector by means of an integral flexural hinge, wherein the mating snap-in element is arranged at the other component out of the locking module and either the line connector or the cannula connector, such that at least one element of the snap-in arrangement is arranged at the locking module and thus at the part to be secured.

The use of a integral flexural hinge for the arrangement of the snap-in element allows, due to the plurality of the degrees of freedom of motion provided by the integral flexural hinge, the forming of a coupling between the snap-in element and the mating snap-in element even when the parts carrying the integral flexural hinge and the mating snap-in element are situation in a position different from a required position for forming the coupling.

In an especially preferred embodiment, the integral flexural hinge, preferably the integral flexural hinge and the snap-in element, is configured as part of the locking module integrally with the locking module. This allows the locking module to be manufactured cost-effectively together with the integral flexural hinge and for example also with the snap-in element in a single step, for example in an injection-molding process. The integral flexural hinge can be configured as a flexible springy tongue with a material bridge to the rest of the locking module. The tongue can in particular be configured through the provision of at least one, preferably two, pass-through slots located in the circumferential direction on both sides of the tongue and proceeding in the flow path direction in a section of the locking module.

In order to further facilitate the handling of the patient therapeutic gas supply system, the patient therapeutic gas supply system can further comprise a pivot lever coupled with the locking module in such a way that by pivoting the pivot lever between a first pivoting region and a second pivoting region the locking module is moved between the locked position and the released position. The use of a pivot lever for effecting the movement of the locking module between the locked position and the released position simplifies, for one thing, the handling of the patient therapeutic gas supply system, and furthermore allows the user to recognize unambiguously whether the locking module is in the locked position or in the released position, depending on whether the pivot lever is in the first pivoting region or the second pivoting region.

The pivot lever can exhibit a dual function, in that additionally to the function just described as an actuation element it also exhibits a function as part of the position-securing device, in that e.g. the snap-in element is arranged at the pivot lever, wherein preferably the mating snap-in element is arranged at the locking module or a connector out of the line connector and the cannula connector.

In order to prevent possible tilting of the locking module during a movement between the locked position and the released position, or at least to reduce the likelihood of same, the patient therapeutic gas supply system comprises a guiding device fixed in place relative to a connector out of the line connector and the cannula connector, which is adapted to guide a movement of the locking module between the locked position and the released position and in particular to restrict it essentially to a translation.

In an especially preferred embodiment, the locking module comprises a closed annular element. As a result, due to the annular form, forces induced in the locking module in the locked position can be distributed symmetrically in the locking module which increases the stability of the locking module. The annular element can comprise or form the locking section.

In addition, it is possible for an annular sleeve of the annular element to exhibit at the front side, i.e. at the end side in the flow path direction, annular element end surfaces, preferably at least one or two annular element end planes, which in particular are each penetrated through by the flow path, and wherein at least one of the annular element end surfaces proceeds at least section-wise non-parallel to a normal plane of the flow path which normal plane intersects the at least one annular element end surface. Such construction allows in particular to match the annular element with the smallest possible material cost to the shape of a positive-locking formation proceeding non-parallel to a normal plane of the flow path between cannula connector or line connector as described above. Preferred are an inclination of the positive-locking formation towards the normal plane of the flow path and a inclination equal as regards magnitude and orientation of the at least one annular element end surface towards the same normal plane of the flow path. Preferably the two annular element end surfaces are parallel to one another.

It is further envisaged that an annular sleeve of the annular element exhibits at the front side, i.e. at the end side in the flow path direction, annular element end surfaces, preferably at least one or two annular element end planes, which in particular are each penetrated through by the flow path, and wherein the annular element end surfaces proceed at least section-wise non-parallel to one another.

Such construction allows, as described above, to guide the annular element along the positive-locking formation, and further allows to provide in the flow path direction a sufficiently large extension of the annular sleeve, at least section-wise with respect to the circumferential direction, in order to accommodate at least parts of functional groups, such as part of the position-securing device, in particular the integral flexural hinge or part of the snap-in device, without increasing the weight of the annular element unnecessarily. Each of these described functional groups can be arranged in a region of a maximum extension of the annular sleeve in the flow path direction, wherein a maximum extension of the annular sleeve in the flow path direction is preferably a genuine maximum extension of the annular sleeve in the flow path direction, such that at another place in the annular sleeve there also exists an extension of the annular sleeve in the flow path direction which is smaller than the maximum extension of the annular sleeve.

The invention will be elucidated below by reference to embodiments, with the help of the attached drawings. They show:

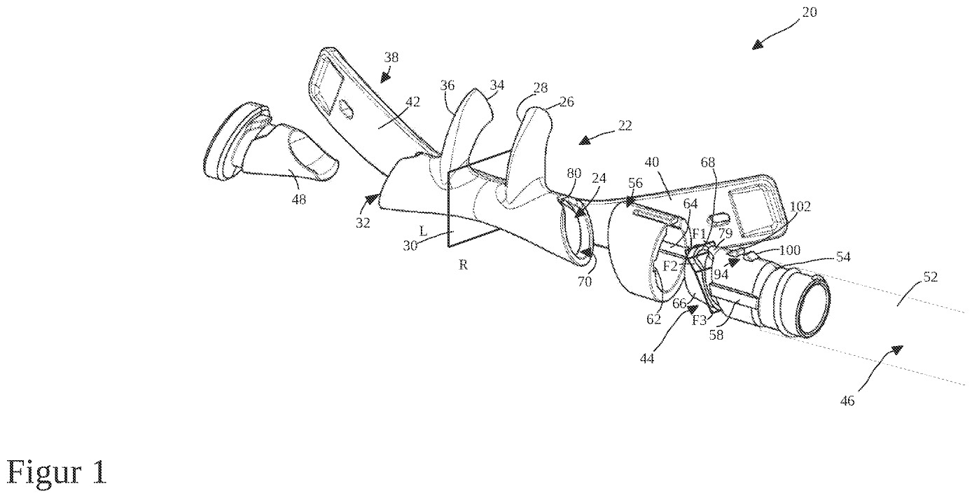

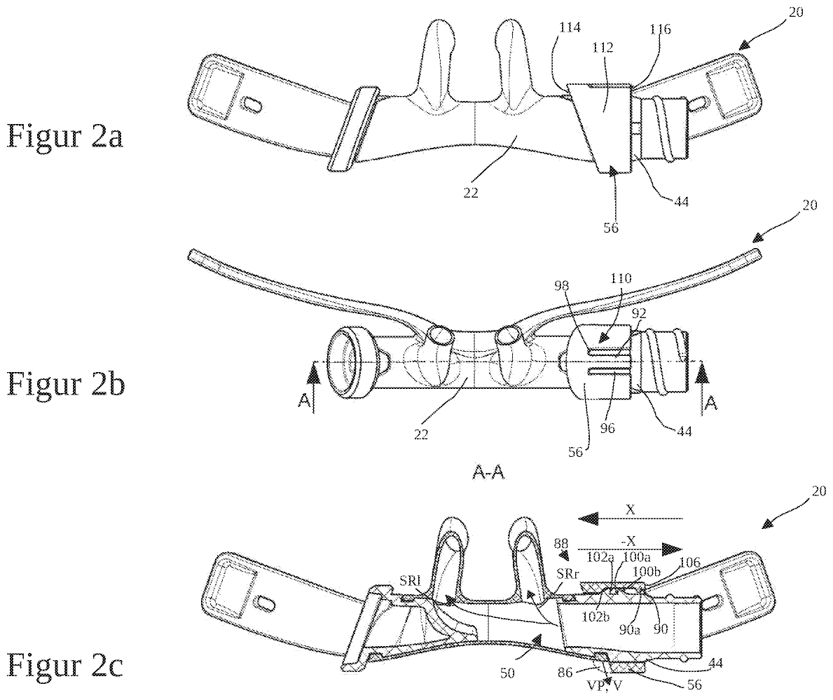

FIG. 1 An exploded view of a first embodiment of the present invention;

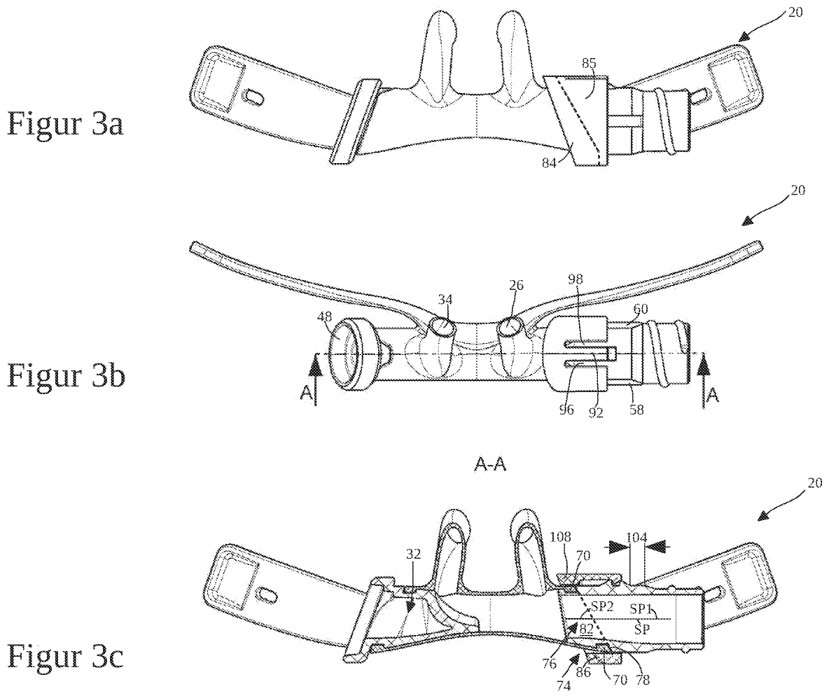

FIG. 2a A side-view of the first embodiment of the present invention from FIG. 1, wherein the locking module is in the released position and wherein parts of the therapeutic gas supply line have been omitted;

FIG. 2b A top view of the first embodiment of the present invention from FIG. 1, wherein the locking module is in the released position shown in FIG. 2a and wherein parts of the therapeutic gas supply line have been omitted;

FIG. 2c The AA cross-section from FIG. 2b;

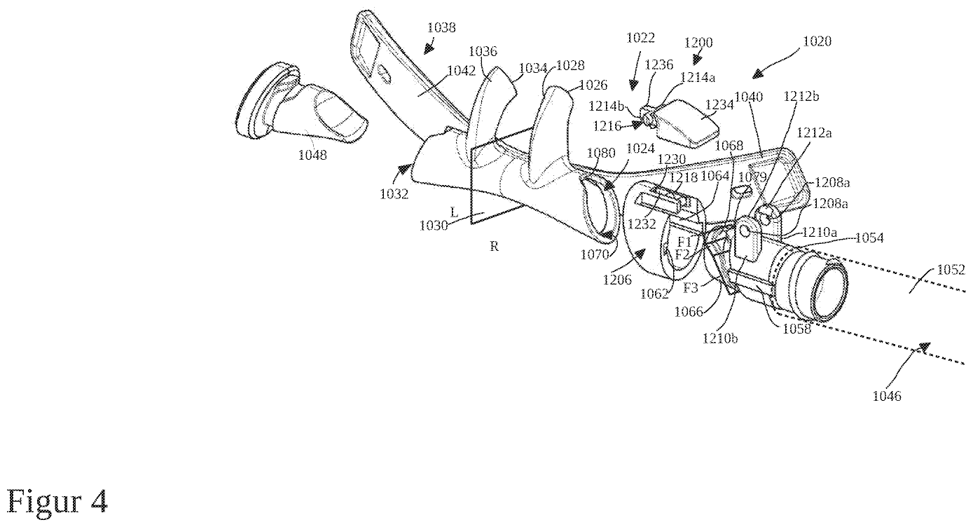

FIG. 3a A side-view of the first embodiment of the present invention from FIG. 1, wherein the locking module is in the locked position and wherein parts of the therapeutic gas supply line have been omitted;

FIG. 3b A top view of the second embodiment of the present invention from FIG. 1, wherein the locking module is in the in the locked position shown in FIG. 3a and wherein parts of the therapeutic gas supply line have been omitted;

FIG. 3c The AA cross-section from FIG. 3b;

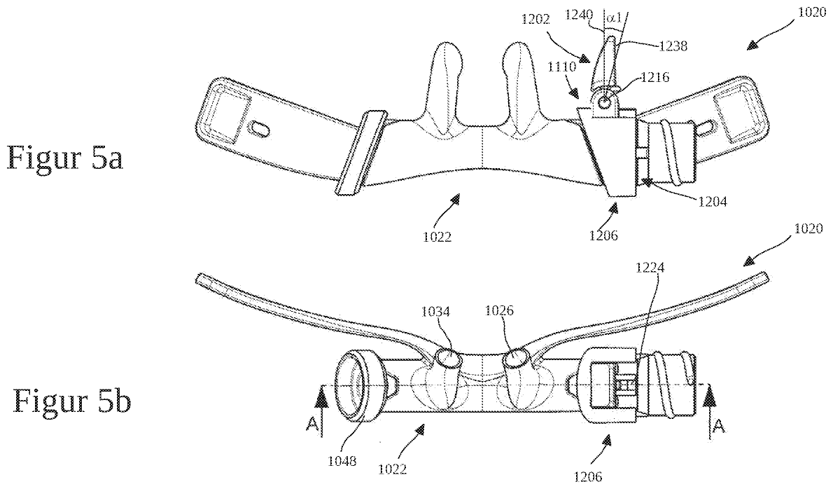

FIG. 4 An exploded view of a second embodiment of the present invention;

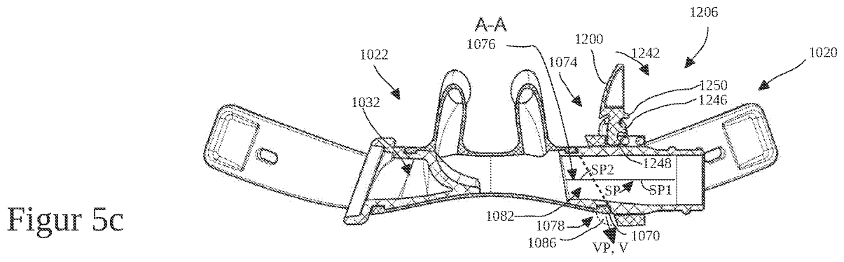

FIG. 5a A side-view of the second embodiment of the present invention from FIG. 4, wherein the locking module is in the released position and wherein parts of the therapeutic gas supply line have been omitted;

FIG. 5b A top view of the second embodiment of the present invention from FIG. 4, wherein the locking module is in the released position and wherein parts of the therapeutic gas supply line have been omitted;

FIG. 5c The AA cross-section from FIG. 5b;

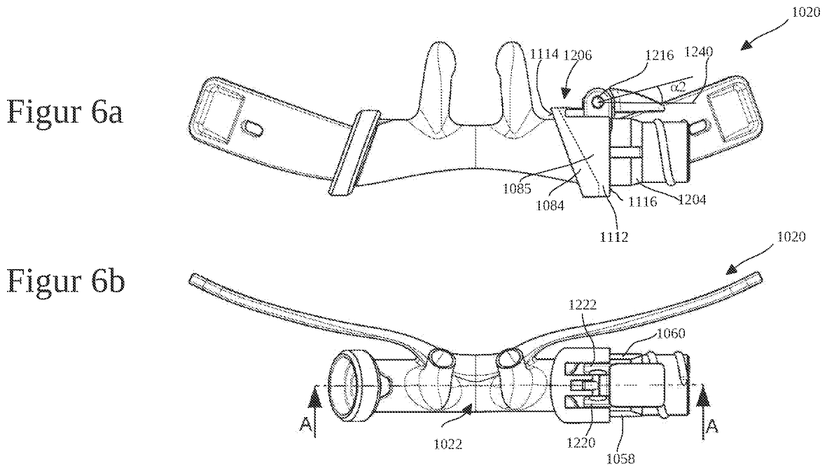

FIG. 6a A side-view of the first embodiment of the present invention from FIG. 4, wherein the locking module is in the locked position and wherein parts of the therapeutic gas supply line have been omitted;

FIG. 6b A top view of the second embodiment of the present invention from FIG. 4, wherein the locking module is in the locked position and wherein parts of the therapeutic gas supply line have been omitted; and

FIG. 6c The AA cross-section from FIG. 6b.

FIG. 1 shows an embodiment according to the invention of a patient therapeutic gas supply system 20 in an exploded view. The patient therapeutic gas supply system 20 comprises a nasal cannula 22, by means of which a patient is supplied with therapeutic gas, for example with air under pressure or with oxygen-enriched air under pressure. The nasal cannula 22 comprises a first cannula connector 24 and a first therapeutic gas-dispensing aperture 26, which can be configured at a first projection 28 of the nasal cannula 22 and via which therapeutic gas is dispensed to the patient. The nasal cannula 22 is preferably configured mirror-symmetrically relative to a plane of symmetry 30 and preferably comprises accordingly due to the mirror symmetry a second cannula connector 32 and a second therapeutic gas-dispensing aperture 34 which can be configured at a second projection 36 of the nasal cannula 22. In addition there can be provided at the nasal cannula 22 a patient-linking arrangement 38 for linking the nasal cannula 22 by means of a strap or a harness to the patient, comprising a first linking arm 40 and a second linking arm 42 and preferably configured mirror-symmetrically relative to the plane of symmetry 30. Due to the mirror symmetry relative to the plane of symmetry 30, in the following only one side R of the nasal cannula 22 will be described and the description should be applied analogously to the side L of the nasal cannula 22 configured mirror-symmetrically relative to the plane 30.

Via a line connector 44 that will be described in detail later on of a therapeutic gas supply line 46 depicted in part by a dotted line of the patient therapeutic gas supply system 20, therapeutic gas can be fed to the nasal cannula 22 via the first cannula connector 24. In order to prevent the therapeutic gas escaping in an unregulated manner through the second cannula connector 32, the latter is closed off with a plug 48; the plug 48 exhibits a gas-guiding surface in order to guide the therapeutic gas flow in the second projection 36 to the second therapeutic gas-dispensing aperture 34, as described in more detail in German patent application DE 10 2018 122 516.4 which had not been released for publication at the filing date. Between the first cannula connector 24 and the two therapeutic gas apertures 26, 34 there is configured a branched fluid conduit 50, wherein in FIG. 2c a flow direction of the therapeutic gas is indicated by arrows SRl, SRr.

The therapeutic gas supply line 46 comprises preferably a hose 52, which is connected to a source of the therapeutic gas at an end not shown, and which at its other end 54 is connected to the line connector 44 preferably via a threaded connector. At the line connector 44 there is arranged a moveable locking module 56 of the patient therapeutic gas supply system 20 formed separately from the former and from the nasal cannula 22 at two guiding projections 58, 60, which engage with grooves 62, 64 configured as complementary in the locking module 56. The guiding projections 58, 60 and the grooves 62, 64 form a guiding device, which guides the movement of the locking module 56 essentially linearly.

The line connector 44 comprises a supporting section 66, at which a groove 68 completely encircling a therapeutic gas-carrying internal space section is configured. At the center of the therapeutic gas-carrying internal space, the therapeutic gas flows from the therapeutic gas source in the direction of the nasal cannula 22 along a flow path sub-path SP1, which in the depicted embodiment can be described inside the line connector 44 by a straight line. The groove 68 does not proceed in a plane which is arranged perpendicularly to the flow path sub-path SP1, but instead its part-regions proceed along a plurality of surfaces F1 to F3, preferably planes F1 to F3, each of which is indicated in FIG. 1. The surfaces F1 and F2 and the surfaces F2 and F3 each intersect and none of the surfaces F1 to F3 proceeds in a plane or parallel to a plane which is arranged perpendicularly to the sub-path SP1 (normal plane to SP1).

At the first cannula connector 24 there is configured a projection 70 protruding inward, which completely encircles a therapeutic gas-carrying internal space section of the first cannula connector 24, in particular a sub-path SP2 in this internal space section, which in the depicted embodiment can be described by a straight line. With the therapeutic gas-carrying connection 74 made, the sub-path SP2 overlays the sub-path SP1 in an overlap region 76 of the cannula connector 22 and the line connector 44. As shown in FIG. 3c, through this overlaying a flow path SP is defined which consists of the sub-path SP2 and the sub-path SP1. The projection 70 is preferably configured as complementary to the groove 68.

The nasal cannula 22 is preferably made from a soft elastic silicone material, whose module of elasticity is smaller than that of the synthetic material from which preferably the line connector 44 is made.

FIGS. 2a-2c and 3a-3c show the patient therapeutic gas supply system 20 in a state in which a therapeutic gas-carrying connection 74 between the first cannula connector 24 and the line connector 44 has been made. In order to make the therapeutic gas-carrying connection 74, the supporting section 66 is inserted into the first cannula connector 24 until the projection 70 as a positive-locking formation snaps into the groove 68 as a positive-locking counter-formation, whereby positive locking of a positive-locking arrangement 78 comprising the groove 68 and the projection 70 is produced.

With a suitable choice of the surfaces F1 to F3, there can be configured at the line connector 44 in a marginal section of the groove 68 a nose 79 protruding outwards beyond the supporting section 66 in a radial direction relative to the sub-path SP1, which when the therapeutic gas-carrying connection 74 has been made protrudes in a direction parallel to the flow path SP into a recess 80 formed complementarily in cannula connector 24. Both the choice of the surfaces F1-F3 and the configuration of the nose 79 and of the recess 80 define an orientation of the nasal cannula 22 relative to the line connector 44, thus forming at least part of an indexing device.

With the therapeutic gas-carrying connection 74 made, the cannula connector 24 and the line connector 44 overlap in a connecting region 82. This region is depicted schematically in FIG. 3c bounded by a dashed line.

FIG. 2c shows that in a released position, the locking module 56 preferably exhibits no overlap with the connecting region 82, whereas in a locked position shown in FIG. 3c the locking module 56 exhibits an overlap with the connecting region 82, which in particular is greater than a possible overlap with the connecting region 82 which can exist when the locking module 56 is in the released position. The flow path SP can be defined also outside the connecting region 82.

If the therapeutic gas-carrying connection 74 is made, then as described above the flow path sub-paths SP1 and SP2 coincide in their overlap region and define in the connecting region 82 in particular the flow path SP, along which the cannula connector 24 and the line connector 44 overlap in the connecting region 82. The statements made about each of the flow path sub-paths SP1 and SP2 apply correspondingly to the flow path SP.

It is preferred especially that in the locked position of the locking module 56 a locking section 84 of the locking module 56 overlaps the positive-locking arrangement 78, this region is depicted schematically in FIG. 3a bounded by a dashed line. Adjacent to the locking section 84, the locking module 56 comprises a carrier section 85, which when the locking module 56 is in the locked position does not overlap with the connecting region.

The locking module 56 can be moved through a relocation, preferably a translation, in a direction X defined by an orientation of the projections 58, 60 from the released position shown in FIG. 2c into the locked position shown in FIG. 3c and can be moved through a relocation, preferably a translation, in an opposite direction −X, which again is defined by the orientation of the projections 58, 60 from the locked position shown in FIG. 3c into the released position.

If the locking module 56 is in the released position and if the nasal cannula 22 is pulled in the direction X and the duct section 44 is pulled in the direction −X, the positive locking formed by the positive-locking arrangement 78 is separated as soon as due to the stretchability of the cannula connector 24 a displacement movement V and/or displacement deformation respectively along a displacement path VP of the projection 70 has moved the projection 70 so far out of the groove 68 that the cannula connector 24 can be separated from the line connector 44, wherein a section of the projection 70 relocated by the displacement movement V forms a displacement section. Consequently, the force needed for separating the cannula connector 24 from the line connector 44 has to overcome the frictional force acting between these two connectors and furthermore provide a force which is induced in the positive-locking arrangement 78 in order to effect the aforementioned displacement movement and/or displacement deformation respectively of the projection 70. In particular, during the displacement movement material of the cannula connector 24 can escape radially outward relative to the flow path SP into a displacement space 86 when the locking module 56 is in the released position.

If the locking module 56 is in the locked position and if the nasal cannula 22 is pulled in the direction X and the duct section 44 is pulled in the direction −X, then at least part of the displacement space 86 is occupied by the locking section 84. Furthermore, the locking section 84 can be configured in the direction of the flow path SP, tapering away from the nasal cannula 22 or with a projection facing radially inwards, such that in the locked position the locking section 84 pre-tensions material of the cannula connector 24 abutting on the line connector 44 in the direction towards the line connector 44 and thus, compared with the released position, contributes to the presence of a quantitatively greater frictional force between the cannula connector 24 and the line connector 44. If the therapeutic gas-carrying connection 74 is separated when the locking module 56 thus configured is in the locked position, then due to the presence of this increased frictional force, the force required for same is greater than when the locking module 56 is in the released position.

Furthermore, material of the cannula connector 24, which when the locking module 56 is in the released position moves into the displacement space 86 during the separating of the therapeutic gas-carrying connection 74, can perform this displacement movement only at least incompletely, since the displacement space 86 is occupied at least in part by the locking section 84. For separating the therapeutic gas-carrying connection 74, the material of the positive-locking arrangement 78 and/or of the locking section 84 has to be deformed more strongly by stretching until the projection 70 has moved so far out of the groove 68 that the positive-locking arrangement 78 is separated. If this stretching is effected by a force which is effected by pulling the nasal cannula 22 in the direction X and pulling the line connector 44 in the direction −X, then due to the chosen geometry and the chosen materials this force is quantitatively greater than in the case where the locking module 56 is in the released position.

If the locking module 56 is situated in the locked position, this constitutes stronger impeding of the displacement movement compared with the case where the locking module 56 is in the released position, since either an additional force has to be exerted in order to deform the material of the locking section 84, in case the displacement movement is carried out completely, or the displacement movement is not carried out completely, which is a special form of impeding the displacement movement.

In order to prevent the locking module 56 exiting the locked position unintentionally, the patient therapeutic gas supply system 20 preferably comprises a position-securing device 88, which comprises a snap-in element configured as a projection 90, which preferably is arranged at a springy tongue 92 forming an integral flexural hinge, and a mating snap-in element configured as a snap-in seat 94. The snap-in element and the mating snap-in element are each part of a snap-in arrangement of the position-securing device 88.

The tongue 92 is configured together with the projection 90 integrally with the locking module 56, wherein in the locking module 56 two pass-through slots 96, 98 are configured in order to allow a bending movement of the tongue 92 and to define its contour at least in part. The snap-in seat 94 is preferably arranged at the line connector 44 and can be configured as a recess, facing radially inward relative to the flow path SP, between two projections 100, 102 spaced apart from each other and arranged at the line connector 44. Each of the projections 100, 102 exhibits a flank 100a, 102a facing towards the other projection and arranged respectively in a normal plane to the flow path SP and a flank 100b, 102b facing away from the other projection which meet an outer surface of the line connector 44 at an angle different from 90°.

The position-securing device 88 is movable between a secured position and a released position. If the locking module 56 is in the locked position and the projection 90 is situated in the snap-in seat 94 between the two projections 100, 102, the position-securing device 88 is in the secured position, whereby the projection 90 is pre-tensioned by the tongue 92 in the position in which the tip of the projection 90 is situated radially further inward relative to the flow path SP than the tip of the projection 100, preferably than the tips of both projections 100, 102. In the secured position, the projection 100 forms for the projection 90, which is configured integrally with the locking module 56, a physical barrier which can be overcome through a movement of the locking module 56 in the direction −X accompanied by deformation of the tongue 92, when a bevel 90a of the projection 90 slides up the projection 100. Once the projection 90 has reached with its tip the tip of the projection 100 during a further movement of the locking module 56 in the direction −X, then the projection 90 slides down the flank 100b. If the projection 90 is situated beyond the flank 100b in the direction −X, in the region 104, then given appropriately chosen dimensioning of the projection 90, optionally only a frictional force acting through the aforementioned pre-tensioning counteracts a movement of the locking module 56 in the direction −X, wherein due the choice of the pre-tensioning and of the module of elasticity of the tongue 92 this frictional force is smaller than the force needed in order to let the bevel 90a of the projection 90 slide up the projection 100. The released position of the position-securing device 88 is reached when the projection 90 has been displaced by a user radially outward relative to the flow path SP so far that neither of the projections 100, 102 interferes in a movement path of the projection 90 in the direction X or −X, such that the elements which form the physical barrier described above in the secured position of the position-securing device 88 do not impact, in particular do not impede, the movement of the locking module 56. In particular, the released position of the locking module 56 is reached when the end 106 of the tongue 92 pointing in the direction −X has reached an end of the region 104 facing away from the snap-in seat 94.

If the projection 90 is situated in the snap-in seat 94, the locking module 56 cannot without an excessive force action separate itself from the patient therapeutic gas supply system 20. If the projection 90 is situated to the right of the projection 100 as shown in FIG. 2c in this view, then an abutting on the flank 102b of a section 108 of the locking module 56 protruding inwards in the radial direction vis-à-vis the positioning of the lug 92 relative to the flow path SP prevents further movement of the locking module 56 in the direction −X. The arrangement of projections thus chosen allows loss-proof arrangement of the locking module 56 at the duct section 44.

The locking module 56 is configured in the embodiment example as a closed annular element surrounding the flow path SP with an annular sleeve 112 which comprises two end-face annular sleeve end planes 114 and 116, which are each penetrated through by the flow path SP. The annular sleeve end planes 114, 116 are not aligned parallel to one another and the annular sleeve end plane 114 is not arranged perpendicular to the flow path SP, such that the annular sleeve end plane 114 cannot be arranged parallel to an arbitrary normal plane of the straight-line-configured flow path SP. The lug 92 is preferably arranged in an extension region 110 of the annular sleeve 112, which exhibits a maximum extension of the annular sleeve 112 in a flow path direction.

A second embodiment of the present invention is described below by reference to FIGS. 4 to 6c, wherein only the differences relative to the first embodiment are discussed.

Parts, sections, regions, directions, etc. which in the second embodiment correspond to those in first embodiment, are labelled with a reference number increased by 1000 and an explicit reference is made to the associated description of the first embodiment, which should also be used for describing the second embodiment as regards the corresponding parts, sections, regions, directions, etc. The reference numbers SP, SP1, and SP2 are also used in the description of the second embodiment and denote paths corresponding to those in the first embodiment, with the same applying to the directions X, −X, the directional information L, R, the arrows SRl, SRr, the displacement path VP, and the displacement movement V. New reference numbers of the second embodiment start at 1200.

The main differences in second embodiment relative to the first embodiment are the provision of a pivot lever 1200, the configuration of the position-securing device 1202 and the resulting dissimilarities in the design of the line connector 1204, and the locking module 1206.

The line connector 1204 exhibits two preferably parallel lugs 1210a, 1210b each provided with an aperture 1208a, 1208b, wherein the apertures 1208a, 1208b are preferably oriented in alignment. At each of the lugs 1210a, 1210b there is preferably provided respectively a lead-in chamfer 1212a, 1212b leading to the associated aperture 1208a, 1208b. The pivot lever 1200 is mounted pivotably in the apertures 1208a, 1208b on an axis 1216 preferably formed by two pin sections 1214a, 1214b. The locking module 1206 is preferably configured as a closed annular element surrounding the flow path SP, whose annular sleeve 1112 extends between the end-face annular sleeve end planes 1114 and 1116. In an extension region 1110 which exhibits a maximum extension of the annular sleeve 1112 in the flow path direction, there is arranged a bridge 1218. A contour of the bridge 1218 is preferably defined by two pass-through slots 1220, 1222 in the circumferential direction relative to the flow path SP. In the bridge there is provided a pass-through slot 1224 along an extension direction of the bridge 1218 preferably parallel to the flow path SP, in which an actuation bridge 1226 and a latching bridge 1228 are arranged movement-proof relative to the bridge 1218. The actuation bridge 1226 and the latching bridge 1228 both extend preferably in a direction transverse to the extension direction of the bridge 1218. Between the actuation bridge 1226 and a blind end 1230 of the pass-through slot 1224 there is provided an accommodating space 1232.

The pivot lever 1200 preferably exhibits an engagement section 1234 for operating by the user and an actuation section 1236, wherein the axis 1216 preferably proceeds through a boundary between the engagement section 1234 and the actuation section 1236. In order to connect the line connector 1204 with the locking module 1206 and the pivot lever 1200, first the locking module 1206 is pushed onto the line connector 1204 starting from the side of the supporting section 1066, such that the bridge 1218 comes to lie between the lugs 1210a, 1210b. Then the pin sections 1214a, 1214b of the pivot lever 1200 are placed at assigned lead-in chamfers 1212a, 1212b, wherein the actuation section 1236 points in the direction towards the nasal cannula 1022. The pivot lever 1200 is moved relative to the flow path SP in a direction oriented radially inward, until the pin sections 1214a, 1214b slide into the assigned apertures 1208a, 1208b at assigned lead-in chamfers 1212a, 1212b, wherein preferably the actuation section 1236 engages in the accommodating space 1232, as shown in FIG. 5c.

For the following angular details, a reference plane 1240 proceeding in parallel to the underside 1238 of the pivot lever 1200 and intersecting the axis 1216 can be used, which in the drawings is indicated by a line. If the reference plane 1240 is in a first pivoting region defined by the angular region α1, as depicted in FIG. 5a, then no or only a small overlap of the locking section 1084 and the positive-locking arrangement 1078 is present, as can be discerned in the depiction in FIG. 5c. If the pivot lever 1200 is pivoted in the pivoting direction 1242 until the reference plane 1240 is situated in the angular region α2 and thus in a second pivoting region, then first during this pivoting a flank 1244 of the actuation section 1236 facing towards the nasal cannula 1022 in FIG. 5b engages with an inner surface of the blind end 1230 and displaces the locking module 1206 from the released position shown in FIG. 5c in the direction of the locked position shown in FIG. 6c, and a first projection 1246 protruding at the pivot lever 1200 engages with the actuation bridge 1226 and pushes over the latter in the further course of the pivoting movement, during which the reference plane 1240 is pivoted into the angular region α2, the locking module 1206 into the locked position show in FIG. 6c. If the pivot lever 1200 is pivoted from the position shown in FIG. 6c and defined by the position of the reference plane 1240 in the angular region α2 against the pivoting direction 1242, then a flank 1248 of the actuation section 1236 facing away from the nasal cannula 1022 engages at the actuation bridge 1226 and displaces the locking module 1206 in the direction of the released position shown in FIG. 5c, which preferably is not reached before the reference plane 1240 is situated in the angular region α1. Through the described interaction of the actuation section 1236 with the actuation bridge 1226 and the inner surface of the blind end 1230, a coupling of the pivot lever 1200 with the locking module 1206 is described. The locked position of the locking module 1206 is preferably not reached before the reference plane 1240 is situated in the angular region α2.

Each of the angular regions α1 and α2 exhibits preferably an angular width of 15° or less.

The position-securing device 1202 comprises the latching bridge 1228 as a snap-in element of a snap-in arrangement, the first projection 1246, and a second projection 1250 protruding at the pivot lever 1200 in the same direction as the first projection 1246, which together form a mating snap-in element of the snap-in arrangement of the position-securing device 1202 assigned to the snap-in element. Preferably, the latching bridge 1228 can only enter the interspace configured between the projections 1246 and 1250 on deformation of one of these projections 1246 and 1250. It is further preferable that as soon as the latching bridge 1228 has entered the interspace between the projections 1246 and 1250, each of the projections 1246 and 1250 takes up again its original position, such that the latching bridge 1228 snaps in or latches in between the projections 1246 and 1250. Accordingly, the pivot lever 1200 is secured in the position shown in FIG. 6c through the described latching mechanism against unintentional rotation opposite to the pivoting direction 1242, which in turn secures the locking module 1206 in the locked position against movement in the direction of the released position due to the engagement of the pivot lever with the line connector 1204 and with the locking module 1206. The position-securing device 1202 further comprises the pivot lever 1200. In the position of the pivot lever 1200 shown in FIG. 6c, the position-securing device 1202 is in the secured position, since via the engagement with the apertures 1208a, 1208b and the engagement with the latching bridge 1228, the pivot lever 1200 forms a physical barrier between the locking module 1206 and the line connector 1206 which cannot be overcome without pivoting or destruction of the pivot lever 1200 or its engagement points with the latching bridge 1228 or the apertures 1208a, 1208b, such that the locking module 1206 cannot be moved into the released position. This is an extreme case of strong impairment of the movement of the locking module 1206 from the locked position to the released position.

If the pivot lever 1200 is pivoted opposite to the pivoting direction 1242 when overcoming the securing force provided by the latching mechanism of the projections 1246 and 1250 at the latching bridge 1228 and the latching mechanism of the projections 1246 and 1250 at the latching bridge 1228 is released, the pivot lever 1200 impedes the movement of the locking module 1206 in the direction of the released position less strongly, since essentially other than a negligible frictional force induced by the rotation of the pin sections 1214a, 1214b in the apertures 1208a, 1208b which has to be overcome during the movement of the locking module 1206 in the direction of the released position of the locking module 1206, the position-securing device 1202 does not create a force acting against movement of the locking module 1206 in the direction of the released position. The force acting on the locking module 1206 that is needed for overcoming the frictional force described here is quantitatively smaller than the force needed for destructive overcoming of the physical barrier provided by the pivot lever 1200 via the engagement with the apertures 1208a, 1208b and the engagement with the latching bridge 1228. In order to determine the forces acting on the locking module 1206, in an adequately good approximation, the pivot lever 1200 can be removed from the apertures 1208a, 1208b and the forces can be measured sufficiently accurately at a thus modified locking module 1206.

Via the latching of the latching bridge 1228 at the projections 1246 and 1250 and the arrangement of the actuation section 1236 in the accommodating space 1232, which optionally cannot be present at the same time, the locking module 1206 is arranged loss-proof at the line connector 1206.

1. A patient therapeutic gas supply system, comprising:
a nasal cannula for the nasal supply of a patient with therapeutic gas, wherein the nasal cannula comprises:
a cannula connector, and
a therapeutic gas-dispensing aperture for dispensing the therapeutic gas to the patient, wherein the nasal cannula defines a fluid conduit between the therapeutic gas-dispensing aperture and the cannula connector;
a therapeutic gas supply line for feeding therapeutic gas to the nasal cannula, wherein the therapeutic gas supply line exhibits a line connector which is adapted to form with the cannula connector a separable therapeutic gas-carrying connection in a connecting region wherein once a therapeutic gas-carrying connection has been made, the cannula connector and the line connector overlap along a section of a flow path proceeding through the connecting region; and
a locking module, wherein the locking module is moveable between a locked position and a released position, wherein a force needed for separating the connection between the cannula connector and the line connector when the locking module is in the locked position is quantitatively larger than when the locking module is in the released position,
characterized in that with the therapeutic gas-carrying connection made, the locking module is displaceable relative to the cannula connector and to the line connector between the locked position and the released position in such a way that in the locked position it exhibits a greater overlap with the connecting region than in the released position.

2. The patient therapeutic gas supply system according to claim 1,
wherein the locking module is configured separately from the cannula connector and the line connector.

3. The patient therapeutic gas supply system according to claim 1,
wherein when the locking module is in the locked position, the locking module at least section-wise pre-tensions a connector out of the cannula connector and the line connector in the direction of the other connector out of the cannula connector and the line connector more strongly than when the locking module is in the released position.

4. The patient therapeutic gas supply system according to claim 1,
Further comprising a positive-locking arrangement which exhibits a positive-locking formation configured at the line connector and a positive-locking ¬counter-formation configured at the cannula connector, wherein the positive-locking formation at least contributes to forming the separable therapeutic gas-carrying connection by forming positive locking between the positive-locking formation and the positive-locking counter-formation;
wherein the positive-locking arrangement is configured in such a way that when the locking module is in the released position, a displacement movement of a displacement section of the positive-locking formation or of the positive-locking counter-formation along a displacement path contributes to separating the positive locking; and
wherein when the locking module is in the locked position, the locking module impedes the displacement movement of the displacement section more strongly than when the locking module is in the released position.

5. The patient therapeutic gas supply system according to claim 4,
wherein a formation out of the positive-locking formation and the positive-locking counter-formation proceeds at least section-wise non-parallel to a normal plane of the flow path which normal plane intersects the one formation out of the positive-locking formation and the positive-locking counter-formation.

6. The patient therapeutic gas supply system according to claim 1, further comprising a position-securing device,
wherein the locking module is securable in the locked position against movement in the direction of the released position by a position-securing device.

7. The patient therapeutic gas supply system according to claim 6,
wherein the position-securing device is movable between a secured position and a released position,
wherein when the locking module is in the locked position and the position-securing device is in the secured position, a physical barrier is formed with the assistance of the position-securing device which impedes movement of the locking module from the locked position into the released position, and
wherein when the position-securing device is in the released position, the physical barrier formed with the assistance of the position-securing device impedes the movement of the locking module from the locked position into the released position less strongly than when the locking module is in the locked position and the position-securing device is in the secured position.

8. The patient therapeutic gas supply system according to claim 6,
wherein the position-securing device comprises a snap-in arrangement with a snap-in element and a mating snap-in element.

9. The patient therapeutic gas supply system according to claim 8,
wherein the snap-in element is arranged at a component out of the locking module and either the line connector or the cannula connector by means of an integral flexural hinge, and
Wherein the mating snap-in element is arranged at the other component out of the locking module and either the line connector or the cannula connector.

10. The patient therapeutic gas supply system according to claim 9,
wherein the integral flexural hinge or the snap-in element and the integral flexural hinge as part of the locking module are configured integrally with the locking module.

11. The patient therapeutic gas supply system according to claim 1, further comprising a pivot lever coupled with the locking module in such a way that through pivoting of the pivot lever between a first pivoting region and a second pivoting region the locking module is moved between the locked position and the released position.

12. The patient therapeutic gas supply system according to claim 1,
wherein the locking module is securable in the locked position against movement in the direction of the released position by a position-securing device,
wherein the position-securing device is movable between a secured position and a released position,
wherein when the locking module is in the locked position and the position-securing device is in the secured position, a physical barrier is formed with the assistance of the position-securing device which impedes movement of the locking module from the locked position into the released position, and
wherein when the position-securing device is in the released position, the physical barrier formed with the assistance of the position-securing device impedes the movement of the locking module from the locked position into the released position less strongly than when the locking module is in the locked position and the position-securing device is in the secured position,
wherein the snap-in element is arranged at the pivot lever and wherein preferably the mating snap-in element is arranged at the locking module or at a connector out of the line connector and the cannula connector.

13. The patient therapeutic gas supply system according claim 1, further comprising a guiding device arranged immovably relative to a connector out of the line connector and the cannula connector, which guiding device is adapted to guide a movement of the locking module between the locked position and the released position and in particular to restrict it essentially to a translation.

14. The patient therapeutic gas supply system according claim 1, wherein the locking module comprises a closed annular element.

15. The patient therapeutic gas supply system according to claim 14,
wherein an annular sleeve of the annular element is bounded by annular element end surfaces, and wherein at least one surface of the annular element end surfaces proceeds at least section-wise non-parallel to a normal plane of the flow path which normal plane intersects the at least one surface of the annular element end surfaces.

16. The patient therapeutic gas supply system according to claim 14,
wherein an annular sleeve of the annular element is bounded by annular element end surfaces, and wherein the annular element end surfaces at least section-wise do not proceed in parallel to one another.

17. The patient therapeutic gas supply system according to claim 14,
wherein an annular sleeve of the annular element is bounded by at least one or two annular element end planes, which are each penetrated through by the flow path, and wherein at least one surface of the annular element end surfaces proceeds at least section-wise non-parallel to a normal plane of the flow path which normal plane intersects the at least one surface of the annular element end surfaces.

18. The patient therapeutic gas supply system according to claim 14,
wherein an annular sleeve of the annular element is bounded by at least one or two annular element end planes, which are each penetrated through by the flow path, and wherein the annular element end surfaces at least section-wise do not proceed in parallel to one another.
